(19) United States
(12) Patent Application Publication
(10) Pub. No.: US 2004/0167439 A1
(43) Pub. Date: Aug. 26, 2004

(54) Guidewire having textured proximal portion

(76) Inventor: James S. Sharrow, Bloomington, MN (US)

Correspondence Address: J. Scot Wickhem, CROMPTON, SEAGER & TUFTE, LLC, 331 Second Avenue South, Suite 895, Minneapolis, MN, 55401-2246, US

(21) Appl. No.: 10/375,633

(22) Filed: Feb. 26, 2003

(51) Int. Cl. A61B 5/00; A61M 25/00

(52) U.S. Cl. 600/585

(57) ABSTRACT

Alternative designs, materials and manufacturing methods for guidewires. Some embodiments pertain to a guidewire having a core wire including a proximal section defining a proximal end and a distal section defining a distal end. The proximal section having a uniform diameter extending from adjacent the proximal end to adjacent the distal section. The distal section having a reduced diameter relative to the proximal section. A tubular polymer layer having an textured outer profile configured to enhance the ability of a user to grip the tubular polymer layer is disposed about a portion of the uniform diameter proximal section of the core wire.

### FIELD OF THE INVENTION

The invention generally pertains to guidewires, and more particularly to guidewires including a textured outer surface about a proximal portion of the guidewire.

### BACKGROUND OF THE INVENTION

A wide variety of guidewires have been developed for medical use, for example intravascular use. Intravascular guidewires are commonly used in conjunction with intravascular devices such as catheters to facilitate navigation through the vasculature of a patient. Because the vasculature of a patient may be very tortuous, it is desirable to combine a number of performance features in a guidewire. For example, it is sometimes desirable that the guidewire have a relatively high level of lubricity to enhance ease of movement within target vessels or within other devices. However, it is also sometimes desirable that an operator of the guidewire be able to grip and control the guidewire, particularly near its proximal end. A number of different guidewire structures and assemblies are known, each having certain advantages and disadvantages. However, there is an ongoing need to provide alternative guidewire structures and assemblies.

### SUMMARY OF THE INVENTION

The invention provides several alternative designs, materials and methods of manufacturing alternative guidewire structures and assemblies.

One embodiment includes a guidewire having a core wire including a proximal section defining a proximal end and a distal section defining a distal end. The proximal section has a uniform diameter extending from adjacent the proximal end to adjacent the distal section. The distal section has a reduced diameter relative to the proximal section. A tubular polymer layer having a textured outer profile configured to enhance the ability of a user to grip the tubular polymer layer is disposed about a portion of the uniform diameter proximal section of the core wire.

Another embodiment provides a guidewire including a core wire having a proximal section defining a proximal end and a distal section defining a distal end. The core wire has a total length defined by the distance between the proximal end and the distal end. A tubular polymer layer having an unsmooth outer profile configured to enhance the ability of a user to grip the tubular polymer layer is disposed about a portion of the proximal section of the core wire. The distal one fifth of the total length of the core wire is free of the tubular polymer layer having an unsmooth outer profile.

Another embodiment provides a medical guidewire configured for use in a patient body, the guidewire having a shaft including a proximal section defining a proximal end and a distal section defining a distal end. The proximal section of the shaft includes a portion that is configured to extend out of the patient's body during use. A tubular polymer layer has a textured outer profile disposed about the portion of the proximal section of the shaft that is configured to extend out of the patient's body during use. The polymer layer outer profile is configured to enhance the ability of a user to grip the tubular polymer layer.

Another embodiment provides a method of forming a guidewire. The method including providing a core wire having a proximal section defining a proximal end and a distal section defining a distal end. The proximal section has a uniform diameter portion extending from adjacent the proximal end to adjacent the distal section. The distal portion has a reduced diameter relative to the proximal portion. A tubular polymer layer has a textured outer surface configured to enhance the ability of a user to grip the polymer layer is disposed around the proximal section of the guidewire.

Another embodiment provides method including inserting a portion of a guidewire into a patient's body and manipulating the guidewire. The guidewire has a shaft including a proximal section defining a proximal end and a distal section defining a distal end. The proximal section of the shaft includes a portion extending out of the patient's body during use. The portion extending out of the patient's body during use includes a polymer layer having a textured outer profile configured to enhance the ability of a user to grip the polymer layer. The guidewire is manipulated by grasping the polymer layer having a textured outer profile and applying torsional or longitudinal force on the polymer layer.

The above summary of some embodiments is not intended to describe each disclosed embodiment or every implementation of the present invention. The Figures, and Detailed Description which follow more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a partial perspective view of a guidewire with a textured proximal portion;

FIG. 2 is a partial cross-sectional view of a guidewire with a textured proximal portion;

FIG. 3 is a cross-sectional view of the guidewire shown in FIG. 1 taken along line 3-3;

FIG. 4 is a perspective view of an alternate embodiment of a textured proximal portion;

FIG. 5 is a partial perspective view of an alternate guidewire with a textured proximal portion;

FIG. 6 is a partial cross-sectional view of an alternate guidewire with a textured proximal portion; and

FIG. 7 is a partial cross-sectional view of an alternate guidewire with a textured proximal portion.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION OF SOME EMBODIMENTS OF THE INVENTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

Weight percent, percent by weight, wt %, wt-%, % by weight, and the like are synonyms that refer to the concentration of a substance as the weight of that substance divided by the weight of the composition and multiplied by 100.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

Refer now to FIG. 1, which is a partial perspective view of one example embodiment of a guidewire 100. The guidewire 100 includes a proximal portion 110 defining a proximal end 111, and a distal portion 115 defining a distal end 116. The proximal portion 110 includes a polymer sleeve 120 having an unsmooth outer surface, and the distal portion 115 includes a coil member 130 and a distal cap 140.

The polymer sleeve 120 having an unsmooth outer surface may extend from the proximal end 111 to a point 112 proximal of the distal end 115 of the guidewire 100. In the embodiment shown, the sleeve 120 extends about the proximal portion 110 of the guidewire 100. The coil 130 extends about the distal portion 115 of the guidewire 100.

The polymer sleeve 120 having an unsmooth outer surface may be disposed over only the proximal portion 110 of the guidewire 100. For example, polymer sleeve 120 may be disposed over up to the proximal 9/10, ⅘, ¾, ⅔, ½, or ¼ of the length of the guidewire 100. In some embodiments, the polymer sleeve 120 may extend to the very proximal end 111 of the guidewire 100, while in other embodiments, the polymer sleeve 120 may end at a point 112 distal of the proximal end 111 of the guidewire 100.

Sleeve 120 may be made of any suitable material including those listed herein. For example, sleeve 120 may be polymeric or otherwise include a polymer. Polymers may include high performance polymers having the desired characteristics such as flexibility, torque-ability, and/or grip-ability. Some examples of suitable polymers may include polytetrafluoroethylene (PTFE) including, for example, expanded PTFE, fluorinated ethylene propylene (FEP), polyurethane, polypropylene (PP), polyvinylchloride (PVC), polyoxymethylene (POM), polybutylene terephthalate (PBT), polyphenylene sulfide (PPS), polyphenylene oxide (PPO), polysufone, perfluroo (propyl vinyl ether) (PFA), polyether-ester (for example a polyether-ester elastomer such as ARNITEL® available from DSM Engineering Plastics), polyester (for example a polyester elastomer such as HYTREL® available from DuPont), polyamide (for example, DURETHAN® available from Bayer or CRISTAMID® available from Elf Atochem), elastomeric polyamides, block polyamide/ethers, polyether block ester, polyether block amide (PEBA, for example available under the trade name PEBAX®), silicones, polyethylene, Marlex high-density polyethylene, linear low density polyethylene (for example REXELL®), polyolefin, polyetheretherketone (PEEK), polyimide (PI), polyetherimide (PEI), nylon, other suitable materials, or mixtures, combinations, copolymers thereof, polymer/metal composites, lubricous polymers, and the like. In some embodiments coating 120 can include a liquid crystal polymer (LCP) blended with other polymers to enhance torqueability. For example, the mixture can contain up to about 5% LCP.

The polymer sleeve 120 can be disposed around and attached to the guidewire 100 using any suitable technique for the particular material used. In some embodiments, the polymer sleeve 120 is attached by heating a sleeve of polymer material to a temperature until it is reformed around the proximal guidewire section 110. In some other embodiments, the polymer sleeve 120 can be attached using heat shrinking techniques. The polymer sleeve 120 may be finished, for example, by a centerless grinding or other method, to provide the desired diameter and to provide an unsmooth outer surface.

The polymer sleeve 120 has an unsmooth or textured surface. This textured surface provides the user of the guidewire 100 with enhanced friction or gripping allowing the user to more easily manipulate the guidewire 100. The textured surface includes a plurality of ridges, splines, or flutes 125 disposed in a longitudinal manner along the length of the guidewire 100, as shown in FIG. 1. The plurality of ridges, splines, or flutes 125 can be disposed about the proximal portion 110 outer perimeter of the guidewire 100. The number of ridges, splines, or flutes 125 can be any number sufficient to enhance friction or gripping such as, for example, 1, 2, 3, 4, 5, 6, 7, 8, 9, 10, 15, 20 or more. The ridges, splines, or flutes 125 can have any height sufficient to enhance friction or gripping such as, for example, 0.005 inch, 0.004 inch, 0.003 inch, 0.002 inch or 0.001 inch. The ridges, splines, or flutes 125 can have any width sufficient to enhance friction or gripping such as, for example, 0.003 inch, 0.005 inch, 0.008 inch or 0.01 inch. Additional friction enhancing coatings may be applied to the polymer sleeve 120.

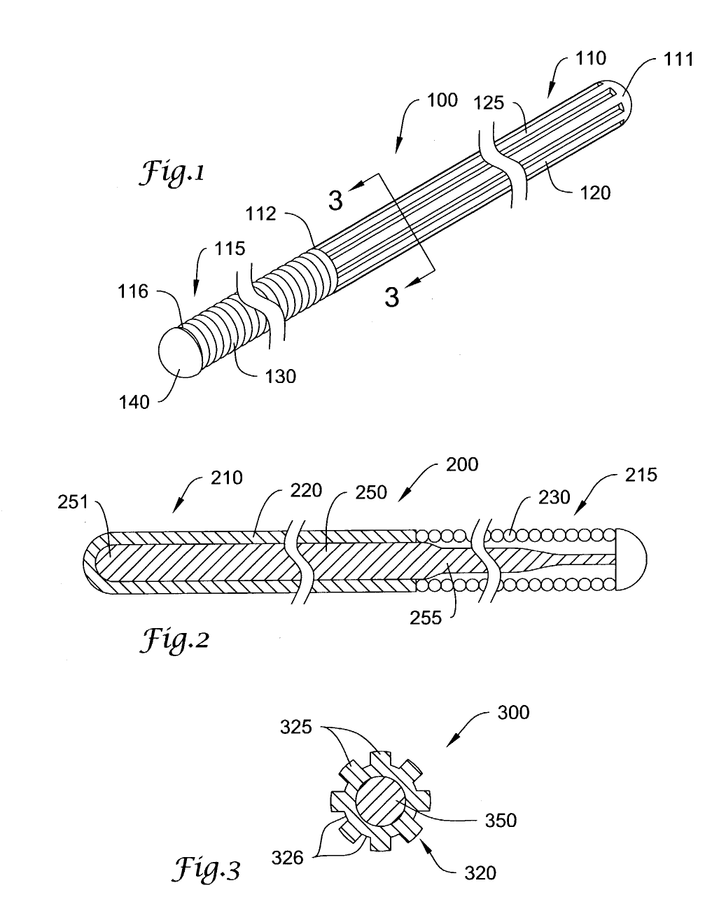

The polymer sleeve 120 may be formed, for example, by coating, by extrusion, co-extrusion, interrupted layer co-extrusion (ILC), fusing or bonding one or more preformed polymer segments to core member 250 (as shown in FIG. 2), or any other appropriate method.

The polymer sleeve 120 may be formed by extruding the polymer sleeve 120 onto the proximal section 110 of the guidewire 100 using an extrusion die that forms the grooves and ridges, splines, or flutes 125 on the outer surface of the sleeve 120 as the polymer sleeve 120 is formed. This type of extrusion can be referred to as “profile extrusion”.

The polymer sleeve 120 may have a uniform stiffness or a gradual reduction in stiffness from the proximal end to the distal end thereof. The gradual reduction in stiffness may be continuous as by ILC or may be stepped as by fusing together separate extruded tubular segments. The polymer sleeve 120 may be impregnated with a radiopaque filler material to facilitate radiographic visualization. Those skilled in the art will recognize that these materials can vary widely without deviating from the scope of the present invention.

A coil 130 may be disposed about the distal portion 115 of the guidewire 100. The coil 130 can be formed of a variety of materials including metals, metal alloys, polymers, and the like. Some examples of material for use in the coil 130 include stainless steel, nickel-chromium alloy, nickel-chromium-iron alloy, cobalt alloy, or other suitable materials. Some additional examples of suitable material include straightened super elastic or linear elastic alloy (e.g., nickel-titanium) wire, or alternatively, a polymer material, such as a high performance polymer. In some embodiments, the coil 130 or portions thereof can be made of or include or be coated with a radiopaque material such as gold, platinum, tungsten, or the like, or alloys thereof. In some embodiments, the coil 130 can be made of a material that is compatible with the core wire 250 (shown in FIG. 2) and the distal cap 140.

The coil 130 can be formed of round or flat ribbon ranging in dimensions to achieve the desired flexibility. In some embodiments, the coil 130 can be a round ribbon in the range of about 0.001-0.015 inches in diameter, and can have a length in the range of about 0.1 to about 20 inches; however, other dimensions are contemplated.

The coil 130 can be wrapped in a helical fashion by conventional winding techniques. The pitch of adjacent turns of the coil 130 may be tightly wrapped so that each turn touches the succeeding turn or the pitch may be set such that the coil 130 is wrapped in an open fashion.

The distal cap 140 can be formed from a variety of different materials, depending on desired performance characteristics. In some embodiments, the distal cap 140 can be formed of a material such as a metallic material that is amenable to being soldered or welded to the distal end 115 of the elongate shaft or core 250, as will be discussed in greater detail hereinafter. In some particular embodiments, it can be beneficial but not necessary for the distal cap 140 to be formed of the same metal or metal alloy as the distal end 115 of the elongate shaft or core 250.

For example, if the elongate shaft or core 250 is formed of stainless steel, it can be beneficial for the distal cap 140 to be formed of stainless steel. In other embodiments, both of the distal cap 140 and the distal end 115 of the elongate shaft or core 250 can be formed of the same metal alloy, such as nitinol. A variety of different processes, such as deep drawing, roll forming or metal stamping can be used to form the distal cap 140. In some embodiments, the distal cap 140 can be metal injection molded. It is contemplated that the distal cap 140 can be formed via a casting process.

A partial cross-sectional view of a guidewire 200 is shown in FIG. 2. The guidewire 200 may include a core wire 250 having a proximal portion 251 and a distal portion 255. Core wire 250 can be made of any suitable materials including metals, metal alloys, polymers, or the like, or combinations or mixtures thereof. Some examples of suitable metals and metal alloys include stainless steel, such as 304v stainless steel; nickel-titanium alloy, such as linear elastic or superelastic (i.e., pseudo elastic) nitinol, nickel-chromium alloy, nickel-chromium-iron alloy, cobalt alloy, tungsten, tungsten alloy, Elgiloy, MP35N, or the like; or other suitable material. The word nitinol was coined by a group of researchers at the United States Naval Ordinance Laboratory (NOL) who were the first to observe the shape memory behavior of this material. The word nitinol is an acronym including the chemical symbol for nickel (Ni), the chemical symbol for titanium (Ti), and an acronym identifying the Naval Ordinance Laboratory (NOL).

Within the family of commercially available nitinol alloys, is a category designated “linear elastic” which, although is similar in chemistry to conventional shape memory and superelastic varieties, exhibits distinct and useful mechanical properties. By skilled applications of cold work, directional stress, and heat treatment, the wire is fabricated in such a way that it does not display a substantial “superelastic plateau” or “flag region” in its stress/strain curve. Instead, as recoverable strain increases, the stress continues to increase in an essentially linear relationship until plastic deformation begins. In some embodiments, the linear elastic nickel-titanium alloy is an alloy that does not show any martensite/austenite phase changes that are detectable by DSC and DMTA analysis over a large temperature range.

For example, in some embodiments, there are no martensite/austenite phase changes detectable by DSC and DMTA analysis in the range of about 60° C. to about 120° C. The mechanical bending properties of such material are therefore generally inert to the effect of temperature over this very broad range of temperature. In some particular embodiments, the mechanical properties of the alloy at ambient or room temperature are substantially the same as the mechanical properties at body temperature. In some embodiments, the use of the linear elastic nickel-titanium alloy allows the guidewire to exhibit superior “push-ability” around tortuous anatomy.

In some embodiments, the linear elastic nickel-titanium alloy is in the range of about 50 to about 60 weight percent nickel, with the remainder being essentially titanium. In some particular embodiments, the composition is in the range of about 54 to about 57 weight percent nickel. One example of a suitable nickel-titanium alloy is FHP-NT alloy commercially available from Furukawa Techno Material Co. of Kanagawa, Japan. In some other embodiments, a superelastic alloy, for example a superelastic nitinol can be used to achieve desired properties. Some examples of nickel-titanium alloys are disclosed in U.S. Pat. Nos. 5,238,004 and 6,508,803, which are incorporated herein by reference.

In at least some embodiments, portions or all of core wire 250 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of guidewire 200 in determining its location. Some examples of radiopaque materials can include, but are not limited to, gold, platinum, palladium, tantalum, tungsten alloy, polymer material loaded with radiopaque filler, and the like.

In some embodiments, a degree of MRI compatibility is imparted into guidewire 200. For example, to enhance compatibility with Magnetic Resonance Imaging (MRI) machines, it may be desirable to make core wire 250, or other portions of the medical guidewire 200, in a manner that would impart a degree of MRI compatibility. For example, core wire 250, or portions thereof, may be made of a material that does not substantially distort the image and create substantial artifacts (artifacts are gaps in the image). Certain ferromagnetic materials, for example, may not be suitable because they may create artifacts in an MRI image. Core wire 250, or portions thereof, may also be made from a material that the MRI machine can image. Some materials that exhibit these characteristics include, for example, tungsten, Elgiloy, MP35N, nitinol, and the like, and others.

The entire core wire 250 can be made of the same material, or in some embodiments, can include portions or sections made of different materials. In some embodiments, the material used to construct core wire 250 is chosen to impart varying flexibility and stiffness characteristics to different portions of core wire 250. For example, proximal portion 251 and distal portion 255 may be formed of different materials, for example materials having different modules of elasticity, resulting in a difference in flexibility. In some embodiments, the material used to construct proximal portion 251 can be relatively stiff for push-ability and torque-ability, and the material used to construct distal portion 255 can be relatively flexible by comparison for better lateral track-ability and steer-ability. For example, proximal portion 251 can be formed of straightened 304v stainless steel wire or ribbon, and distal region 215 can be formed of a straightened super elastic or linear elastic alloy, for example a nickel-titanium alloy wire or ribbon.

In embodiments where different portions of core wire 250 are made of different material, the different portions can be connected using any suitable connecting techniques. For example, the different portions of the core wire 250 can be connected using welding, soldering, brazing, adhesive, or the like, or combinations thereof. Additionally, some embodiments can include one or more mechanical connectors or connector assemblies to connect the different portions of the core wire that are made of different materials. The connector may include any structure generally suitable for connecting portions of a guidewire. One example of a suitable structure includes a structure such as a hypotube or a coiled wire which has an inside diameter sized appropriately to receive and connect to the ends of the proximal portion and the distal portion. Some other examples of suitable techniques and structures that can be used to interconnect different shaft sections are disclosed in U.S. patent application Ser. No. 09/972,276 filed on Oct. 5, 2001 and Ser. No. 10/068,992 filed on Feb. 28, 2002, which are incorporated herein by reference. Some other examples are disclosed in a U.S. Patent Application entitled “COMPOSITE MEDICAL DEVICE” (Attorney docket no. 1001.1546101) filed on even date herewith, which is incorporated herein by reference. Some other examples are disclosed in a U.S. Patent Application entitled “ARTICULATING INTRACORPORAL MEDICAL DEVICE” (Attorney docket no. 1001.1668101) filed on even date herewith, which is incorporated herein by reference.

The length of core member 250 (and/or guidewire 200), or the length of individual portions thereof, are typically dictated by the length and flexibility characteristics desired in the final medical device. For example, proximal portion 210 may have a length in the range of about 20 to about 300 centimeters or more and distal portion 215 may have a length in the range of about 3 to about 50 centimeters or more. It can be appreciated that alterations in the length of portions 210/215 can be made without departing from the spirit of the invention.

Core wire 250 can have a solid cross-section, but in some embodiments, can have a hollow cross-section. In yet other embodiments, core wire 250 can include a combination of areas having solid cross-sections and hollow cross sections. Moreover, core 250, or portions thereof, can be made of rounded wire, flattened ribbon, or other such structures having various cross-sectional geometries. The cross-sectional geometries along the length of core 250 can also be constant or can vary. For example, FIG. 2 depicts core wire 250 as having a round cross-sectional shape. It can be appreciated that other cross-sectional shapes or combinations of shapes may be utilized without departing from the spirit of the invention. For example, the cross-sectional shape of core wire 250 may be oval, rectangular, square, polygonal, and the like, or any suitable shape.

As shown in FIG. 2, distal portion 255 can include one or more tapers or tapered regions that reduce the core 250 in size or diameter. For example, in some embodiments, distal portion 255 can have an initial outside diameter that is in the range of about 0.010 to about 0.040 inches, than tapers to a diameter in the range of about 0.001 to about 0.01 inches. The tapered regions may be linearly tapered, tapered in a curvilinear fashion, uniformly tapered, non-uniformly tapered, or tapered in a step-wise fashion. The angle of any such tapers can vary, depending upon the desired flexibility characteristics. The length of the taper may be selected to obtain a more (longer length) or less (shorter length) gradual transition in stiffness. As shown in FIG. 2, the tapered region may include one or more portions where the outside diameter is narrowing, for example, the tapered portions, and portions where the outside diameter remains essentially constant, for example, constant diameter portions. The number, arrangement, size, and length of the narrowing and constant diameter portions can be varied to achieve the desired characteristics, such as flexibility and torque transmission characteristics. The narrowing and constant diameter portions as shown in FIG. 2 are not intended to be limiting, and alterations of this arrangement can be made without departing from the spirit of the invention.

The tapered and constant diameter portions of the tapered region may be formed by any one of a number of different techniques, for example, by centerless grinding methods, stamping methods, and the like. The centerless grinding technique may utilize an indexing system employing sensors (e.g., optical/reflective, magnetic) to avoid excessive grinding of the connection. In addition, the centerless grinding technique may utilize a CBN or diamond abrasive grinding wheel that is well shaped and dressed to avoid grabbing core wire during the grinding process. In some embodiments, core wire 250 can be centerless ground using a Royal Master HI-AC centerless grinder. Some examples of suitable grinding methods are disclosed in U.S. patent application Ser. No. 10/346,698 filed Jan. 17, 2003, which is herein incorporated by reference.

The textured polymer sleeve 220 is disposed on the proximal portion 210 of the guidewire 200. The proximal portion 210 of the guidewire 200 in this particular embodiment can be defined as being the portion of the guidewire 200 where the core wire 250 has a relatively uniform size or diameter and may be the largest size or diameter along the core 250. The distal portion 215 of the guidewire 200 in this particular embodiment can be defined as being the portion of the guidewire 200 where the core wire 250 reduces in size from the relatively uniform diameter proximal portion 210 in the form of tapers or the like. It is understood that in other embodiments the proximal 210 and distal 215 sections of the guidewire 200 can be defined differently for example, in terms of total length or length relative to one another, in terms of stiffness or flexibility characteristics or other structural elements.

In some other embodiments, a polymer jacket tip or combination coil/polymer tip, and other such structure, such as radiopaque markers, safety and/or shaping ribbons (coiled or uncoiled), and the like, may be placed on the guidewire 200. Additionally, in some embodiments, a coating, for example a lubricious (e.g., hydrophylic) or other type of coating may be applied over portions or all of the polymeric sleeve 220, coil 230, or other portions of the guidewire 200. Hydrophobic coatings such as fluoropolymers provide a dry lubricity which improves guide wire handling and device exchanges. Lubricious coatings improve steerability and improve lesion crossing capability. Suitable lubricious polymers are well known in the art and may include hydrophilic polymers such as polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

FIG. 3 is a cross-sectional view of the guidewire 100 shown in FIG. 1 taken along line 3-3. The guidewire 300 includes a core 350 as described above and a textured sleeve 320 as described above. The textured sleeve 320 includes a plurality of ridges, splines, or flutes 325 that enhance the ease of use of the guidewire as described above. The textured sleeve 320 includes a plurality of grooves 326. Each groove 326 can be disposed between two ridges, splines, or flutes 325. Alternatively, each ridge, spline, or flute 325 can be disposed between two grooves 326. Each groove 326 may span a width between ridges, splines or flutes 325 from 0.001 inch to 0.01 inch or from 0.003 inch to 0.006 inch.

FIG. 4 is a perspective view of an alternate embodiment of a textured proximal portion 400. The textured surface includes a plurality of protrusions 425 disposed in a longitudinal and circumferential manner along the length of the textured proximal portion 400. The plurality of protrusions 425 can be disposed in a uniform (as shown) or non-uniform manner or pattern along the length of the proximal portion. For example, the density of protrusions may increase or decrease along the length of the proximal portion 400. The number of protrusions 425 can be any number sufficient to enhance friction or grip such as, for example, 1, 2, 3, 4, 5, 6, 7, 8, 9, 10, 15, 20, 50, 100, 200 or more. The protrusions 425 can have any height sufficient to enhance friction such as, for example, 0.005 inch to 0.01 inch. The protrusions 425 can have any width sufficient to enhance friction such as, for example, 0.005 inch to 0.01 inch. The protrusions 425 can have a constant height or width along the proximal portion 400 or the height and width of each protrusion 425 can increase or decrease along the proximal portion 400. For example, the height of the protrusions 425 may decrease along the proximal portion 400 to allow for a greatly enhanced frictional surface proximate to the very proximal end of a guidewire. Additional friction enhancing coatings may be applied to the proximal portion 400.

While protrusions 425 have been shown as rectangular in shape, it is contemplated that the protrusions may be any shape including, for example, round, domed, triangular, pyramidal, oval, diamond, or randomly shaped.

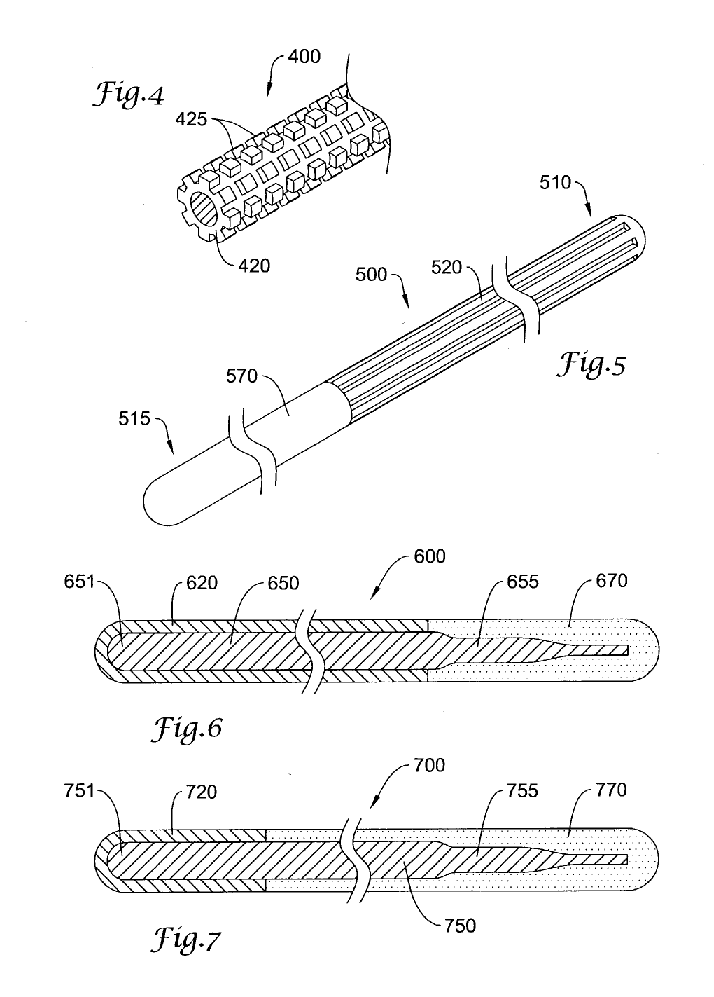

FIG. 5 is a partial perspective view of an alternate guidewire 500 with a textured proximal portion 520. A polymer sheath 570 is disposed over a distal portion of the guidewire 500. FIG. 6 is a partial cross-sectional view of the guidewire 500 shown in FIG. 5. The textured proximal sleeve 520, 620 is disposed over the proximal portion 651 of the core 650. The polymer sheath 570, 670 is disposed over the distal portion 655 of the core 650.

In this embodiment a polymer tip guidewire 500, 600 is formed by including the polymer sheath 570, 670 that forms a rounded tip over the distal portion 655 of the core 650. The polymer sheath 570 can be made from any material that can provide the desired strength, flexibility or other desired characteristics. The polymer sheath 570 can in some non-limiting embodiments have a length that is in the range of 2 cm to 100 cm and can have an inner diameter that is in the range of about 0.002 inch to 0.03 inch and an outer diameter that is in the range of about 0.01 inch to 0.04 inch.

The use of a polymer can serve several functions, such as improving the flexibility properties of the guidewire assembly. Choice of polymers for the sheath or sleeve 570 will vary the flexibility of the guidewire. For example, polymers with a low durometer or hardness will make a very flexible or floppy tip. Conversely, polymers with a high durometer will make a tip which is stiffer. The use of polymers for the sleeve can also provide a more atraumatic tip for the guidewire. An atraumatic tip is better suited for passing through fragile body passages. Finally, a polymer can act as a binder for radiopaque materials, as discussed in more detail below.

Some suitable materials include polymers, and like material. Examples of suitable polymer material include any of a broad variety of polymers generally known for use as guidewire polymer sleeves. In some embodiments, the polymer material used is a thermoplastic polymer material. Some examples of some suitable materials include polyurethane, elastomeric polyamides, block polyamide/ethers (such as Pebax), silicones, and co-polymers. The sleeve may be a single polymer, multiple layers, or a blend of polymers. By employing careful selection of materials and processing techniques, thermoplastic, solvent soluble and thermosetting variants of these materials can be employed to achieve the desired results.

Further examples of suitable polymeric materials include but are not limited to poly(L-lactide) (PLLA), poly(D,L-lactide) (PLA), polyglycolide (PGA), poly(L-lactide-co-D,L-lactide) (PLLA/PLA), poly(L-lactide-co-glycolide) (PLLA/PGA), poly(D,L-lactide-co-glycolide) (PLA/PGA), poly(glycolide-co-trimethylene carbonate) (PGA/PTMC), polyethylene oxide (PEO), polydioxanone (PDS), polycaprolactone (PCL), polyhydroxylbutyrate (PHBT), poly(phosphazene), poly D,L-lactide-co-caprolactone) (PLA/PCL), poly(glycolide-co-caprolactone) (PGA/PCL), polyanhydrides (PAN), poly(ortho esters), poly(phosphate ester), poly(amino acid), poly(hydroxy butyrate), polyacrylate, polyacrylamid, poly(hydroxyethyl methacrylate), polyurethane, polysiloxane and their copolymers.

In some embodiments, the sheath 570, 670 or portions thereof, can include, or be doped with, radiopaque material to make the sheath 570, 670 or portions thereof, more visible when using certain imaging techniques, for example, fluoroscopy techniques. Any suitable radiopaque material known in the art can be used. Some examples include precious metals, tungsten, barium subcarbonate powder, and the like, and mixtures thereof. In some embodiments, the polymer can include different sections having different amounts of loading with radiopaque material. For example, the sheath or sleeve 570 can include a distal section having a higher level of radiopaque material loading, and a proximal section having a correspondingly lower level of loading.

In some embodiments, it is also contemplated that a separate radiopaque member or a series of radiopaque members, such as radiopaque coils, bands, tubes, or other such structures could be attached to the guidewire core wire 650 or incorporated into the core wire by plating, drawing, forging, or ion implantation techniques.

The sheath 570, 670 can be disposed around and attached to the guidewire assembly 500, 600 using any suitable technique for the particular material used. In some embodiments, the sheath 570, 670 can be attached by heating a sleeve of polymer material to a temperature until it is reformed around the guidewire assembly 500, 600. In some other embodiments, the sheath 570, 670 can be attached using heat shrinking techniques. In other embodiments, the sheath or sleeve 570, 670 can be co-extruded with the core wire 650. The sheath 570, 670 can be finished, for example, by a centerless grinding or other method, to provide the desired diameter and to provide a smooth outer surface.

FIG. 7 is a partial cross-sectional view of an alternate guidewire 700 with a textured proximal portion. FIG. 7 is similar to FIG. 6 except that the textured polymer sleeve 720 is disposed over only a portion of the proximal portion 751 of the core 750. The polymer sheath 770 is disposed over the distal portion 755 of the core 750 and a portion of the proximal portion 751.

The medical device described herein is configured to extend out of the patient's body during use. The portion of the medical device not in the patient's body during use includes the textured or unsmooth polymer sleeve. The textured or unsmooth polymer sleeve is configured to enhance the ability of a user to grip the textured polymer sleeve for procedures. The textured polymer sleeve improves the user's ability to manipulate the medical device such as, for example, a guidewire. The textured polymer sleeve improves the user's ability to push the medical device into the patient's body and improves the user's ability to rotate the medical device once the medical device in placed in the patient's body.

The present invention should not be considered limited to the particular examples described above, but rather should be understood to cover all aspects of the invention as fairly set out in the attached claims. Various modifications, equivalent processes, as well as numerous structures to which the present invention may be applicable will be readily apparent to those of skill in the art to which the present invention is directed upon review of the instant specification. It should be understood that this disclosure is, in many respects, only illustrative. Changes may be made in details, particularly in matters of shape, size, and arrangement of steps without exceeding the scope of the invention. The scope of the invention is, of course, defined in the language in which the appended claims are expressed.

What is claimed is:

1. A guidewire, comprising:
a core wire including a proximal section defining a proximal end and a distal section defining a distal end, the proximal section having a uniform diameter extending from adjacent the proximal end to adjacent the distal section, the distal section having a reduced diameter relative to the proximal section; and
a tubular polymer layer having an textured outer profile configured to enhance the ability of a user to grip the tubular polymer layer, the tubular polymer layer being disposed about a portion of the uniform diameter proximal section of the core wire.

2. The guidewire according to claim 1, wherein the textured outer profile includes a plurality of longitudinally extending and circumferentially spaced ridges, splines, or flutes.

3. The guidewire according to claim 1, wherein the textured outer profile includes a plurality of protrusions.

4. A guidewire, comprising:
a core wire including a proximal section defining a proximal end and a distal section defining a distal end, the core wire having a total length defined by the distance between the proximal end and the distal end;
a tubular polymer layer having an unsmooth outer profile configured to enhance the ability of a user to grip the tubular polymer layer, the tubular polymer layer being disposed about a portion of the proximal section of the core wire, wherein the distal one fifth of the total length of the core wire is free of the tubular polymer layer having an unsmooth outer profile.

5. The guidewire according to claim 4, wherein the distal one quarter of the total length of the core wire is free of the polymer layer having an unsmooth outer profile.

6. The guidewire according to claim 4, wherein the distal one half of the total length of the core wire is free of the polymer layer having an unsmooth outer profile.

7. The guidewire according to claim 4, wherein the distal three quarters of the total length of the core wire is free of the polymer layer having an unsmooth outer profile.

8. A medical guidewire configured for use in a patient body, the guidewire comprising:
a shaft including a proximal section defining a proximal end and a distal section defining a distal end, the proximal section of the shaft including a portion that is configured to extend out of the patient's body during use;
a tubular polymer layer having a textured outer profile disposed about the portion of the proximal section of the shaft that is configured to extend out of the patient's body during use, the polymer layer outer profile being configured to enhance the ability of a user to grip the tubular polymer layer.

9. The medical guidewire according to claim 8, wherein the textured outer profile includes a plurality of longitudinally extending and circumferentially spaced ridges, splines, or flutes.

10. The medical guidewire according to claim 8, wherein the textured outer profile includes a plurality of protrusions.

11. A method of forming a guidewire, the method comprising:
providing a core wire including a proximal section defining a proximal end and a distal section defining a distal end, the proximal section including a uniform diameter portion extending from adjacent the proximal end to adjacent the distal section, the distal portion having a reduced diameter relative to the proximal portion;
disposing a tubular polymer layer having an textured outer surface configured to enhance the ability of a user to grip the polymer layer around the proximal section of the guidewire.

12. The method according to claim 11, wherein the tubular polymer layer is disposed around the proximal section of the guidewire through profile coextrusion.

13. The method according to claim 11, wherein the textured surface includes a plurality of longitudinally extending and circumferentially spaced ridges, splines, or flutes.

14. The method according to claim 11, wherein the textured surface includes a plurality of protrusions.

16. The method according to claim 11, wherein the textured surface is disposed on up to ¾ of the proximal portion of the guidewire.

17. The method according to claim 11, wherein the textured surface is disposed on up to ½ of the proximal portion of the guidewire.

18. The method according to claim 11, wherein the textured surface is disposed on up to ⅓ of the proximal portion of the guidewire.

19. A method of forming a guidewire, the method comprising:
providing a core wire including a proximal section defining a proximal end and a distal section defining a distal end, the core wire having a total length defined by the distance between the proximal end and the distal end;
disposing a tubular polymer layer having an unsmooth outer profile configured to enhance the ability of a user to grip the tubular polymer layer about a portion of the proximal section of the core wire, wherein the distal one fifth of the total length of the core wire is free of the tubular polymer layer having an unsmooth outer profile.

20. The guidewire according to claim 19, wherein the distal one quarter of the total length of the core wire is free of the polymer layer having an unsmooth outer profile.

21. The guidewire according to claim 19, wherein the distal one half of the total length of the core wire is free of the polymer layer having an unsmooth outer profile.

22. The guidewire according to claim 19, wherein the distal three quarters of the total length of the core wire is free of the polymer layer having an unsmooth outer profile.

23. A method comprising:
inserting a portion of a guidewire into a patient's body, the guidewire having a shaft including a proximal section defining a proximal end and a distal section defining a distal end, the proximal section of the shaft including a portion extending out of the patient's body during use; wherein the portion extending out of the patient's body during use includes a polymer layer having a textured outer profile configured to enhance the ability of a user to grip the polymer layer;
manipulating the guidewire by grasping the polymer layer having a textured outer profile and applying torsional or longitudinal force on the polymer layer.

24. The method according to claim 23, wherein the textured outer profile includes a plurality of longitudinally extending and circumferentially spaced ridges, splines, or flutes.

25. The according to claim 23, wherein the textured outer profile includes a plurality of protrusions.

26. A product produced by the method of claim 11.

27. A product produced by the method of claim 19.
